(19) United States
(12) Patent Application Publication
(10) Pub. No.: US 2014/0128402 A1
(43) Pub. Date: May 8, 2014

(54) PHARMACEUTICAL COMBINATION

(71) Applicant: IPCA Laboratories Limited, Mumbai (IN)

(72) Inventors: Anil PAREEK, Mumbai (IN); Gopakumar G. NAIR, Mumbai (IN)

(73) Assignee: IPCA Laboratories Limited, Mumbai (IN)

(21) Appl. No.: 14/152,568

(22) Filed: Jan. 10, 2014

Related U.S. Application Data
(63) Continuation-in-part of application No. PCT/IN2012/000491, filed on Jul. 11, 2012.

(30) Foreign Application Priority Data
Jul. 12, 2011 (IN) 2002/MUM/2011

(51) Int. Cl. A61K 31/4706; A61K 31/522; A61K 31/403; A61K 31/4985; A61K 31/40

(52) U.S. Cl. CPC A61K 31/4706; A61K 31/4985; A61K 31/40; A61K 31/403; A61K 31/522
USPC 514/249; 514/313; 514/263.21

(57) ABSTRACT

The present invention discloses a pharmaceutical combination comprising Hydroxychloroquine and a DPP-IV inhibitor or their pharmaceutically acceptable salts, solvates or prodrugs thereof, for preventing, slowing the progression of, delaying, improving, restoring, or treating a condition or a disease resulting from metabolic disorders.

This application is a continuation-in-part (CIP) of PCT/IN2012/000491, filed Jul. 11, 2012, which claims the priority of Indian Patent Application No. 2002/MUM/2011, filed Jul. 12, 2011, which are incorporated herein by reference.

### TECHNICAL FIELD OF THE INVENTION

The present invention relates to a pharmaceutical combination useful for the treatment of metabolic syndrome. The invention also relates to a method for the treatment of said metabolic disorders/diseases, comprising simultaneous, separate or sequential administration of effective amounts of specific active compounds and/or co-treatment, in a ratio which provides an additive and/or synergistic effect, and to the combined use of these specific compounds for the manufacture of corresponding pharmaceutical combination preparations. The invention relates more specifically to a pharmaceutical combination comprising Hydroxychloroquine and a DPP-IV inhibitor or their pharmaceutically acceptable salts, solvates or prodrugs thereof, for the treatment of Type-2 diabetes mellitus.

### BACKGROUND OF THE INVENTION

According to the American Heart Association, about 60% of male and nearly 50% of female are overweight, in which approximately 18% are relatively obese. Obesity can lead to metabolic syndrome, apart from other factors, which is characterized by a group of metabolic risk factors in one person. They include: (a) central obesity, indicated by excessive fat tissue in and around the abdomen; (b) dyslipidemia (blood fat disorders, mainly high triglycerides and low HDL cholesterol, that foster plaque buildups in artery walls); (c) elevated blood pressure; (d) insulin resistance or glucose intolerance (the body can't properly use insulin or blood sugar); (e) prothrombotic state (e.g., high fibrinogen or plasminogen activator inhibitor in the blood); and (f) pro-inflammatory conditions.

The underlying causes of metabolic disorders/syndrome may include overweight/obesity, physical inactivity and genetic factors. People with metabolic syndrome are at increased risk of coronary heart disease, other diseases related to plaque buildup in artery walls (e.g., stroke and peripheral vascular disease) and Type 2 diabetes. According to the American Diabetes Association, 20.6% of adults over the age of 60 have diabetes and 34.8% of all adults have either diabetes or pre-diabetes. Metabolic syndrome has become increasingly common in the western world. The syndrome is closely associated with a generalized metabolic disorder called insulin resistance, in which the body cannot use insulin efficiently. Metabolic syndrome is also called insulin resistance syndrome, which leads to Type 2 diabetes.

Non-insulin dependent diabetes mellitus (type 2 diabetes mellitus) is characterized by both increased peripheral insulin resistance and abnormal insulin secretion. At least three abnormalities of insulin secretion are recognized: in the first phase, insulin secretion is lost and in the second phase insulin is both delayed and inadequate in the face of elevated circulating glucose levels. Several metabolic, hormonal, and pharmacological entities are known to stimulate insulin secretion including glucose, amino-_acids and gastrointestinal peptides. The Diabetes Control and Complications Trial (DCCT) have established that lowering of blood glucose is associated with decreases in the onset and progression of diabetic microvascular complications (Diabetes Control and Complications Trial Research Group; N. Engl. J. Med. 1993, 329, 977-986) Impaired Glucose Tolerance is an impairment of glucose homeostasis closely related to type 2 diabetes mellitus. Both conditions convey a great risk of macrovascular disease. Therefore, one therapeutic focus is on optimizing and potentially normalizing glycemic control in subjects with type 2 diabetes mellitus, conditions of impaired fasting plasma glucose, or IGT.

Glucose-dependent insulinotropic polypeptide (GIP) and glucagon-like peptide (GLP-1) are the 2 major incretin hormones released after meals to enhance glucose-stimulated insulin secretion and control the increase in glucose levels. In patients with type 2 diabetes, a loss of activity of GIP for insulinotropic function and a reduced secretion of GLP-1 exist in response to oral glucose while GLP-1 action is preserved. GLP-1 is therefore an attractive avenue for treating type 2 diabetes. GLP-1 is degraded by dipeptidyl peptidase IV (DPP-IV). Dipeptidyl Peptidase-IV (DPP-IV) inhibiting agents are of particular pharmacological significance, and represent a novel class of oral antihyperglycemic agents for the treatment of type 2 diabetes. Specific DPP-4 inhibitors either already approved for marketing or under clinical development for the treatment of Type 2 diabetes include sitagliptin, vildagliptin, saxagliptin, melogliptin, P93/01 (Prosidion), alogliptin, denagliptin, Roche 0730699, TS021 (Taisho), and E3024 (Eisai), linagliptin, carmegliptin, gosogliptin, teneligliptin and dutogliptin. For example, oral administration of sitagliptin, vildagliptin, alogliptin, and saxagliptin to human Type 2 diabetics has been found to reduce fasting glucose and postprandial glucose excursion in association with significantly reduced HbA1c levels. The therapeutic utility of these antihyperglycemic agents rest on their ability to increase active (intact) levels of incretin peptides, including glucagon-like peptide-1 (GLP-1) and glucose-dependent insulinotropic peptide (GIP).

Hydroxychloroquine, a commonly used antirheumatic medication, has hypoglycemic effect and may reduce the risk of diabetes. The previous studies in diabetic rats show that hydroxychloroquine significantly elevates insulin concentration in the blood resulting in reduced blood glucose. It is also reported to inhibit insulin metabolism by inhibiting cytosolic insulin metabolizing enzyme. Clinically, hydroxychloroquine showed improvement in sulphonylurea refractory patients with poorly controlled type 2 diabetes. Along with anti-hyperglycemic effects, hydroxychloroquine is also associated with cardiovascular benefits. Clinical studies show that changes in lipids with hydroxychloroquine may lead to a significant reduction in IHD risk.

However, in more than 10% of patients glycaemic control with monotherapy cannot be maintained. Since the disease itself is progressive and the therapeutic attempts to achieve and maintain glycemic control often fail in the long run, type 2 diabetic patients are required to be treated with a combination of therapies/drugs. Metabolic disorders lead to a cascade of aforementioned diseases and it is always advantageous if oral treatment for diabetes mellitus would be a treatment that not only controls the glycemic level, but also prevents the development of atherosclerosis and other complications associated with metabolic disorders.

The object of the present invention is to develop pharmaceutical drug products for the treatment of metabolic disorders which ameliorate at least one symptom of the condition/disease or improves the condition thereof, if not all, in the treatment of diseases arising out of metabolic syndrome, especially diabetes mellitus.

### SUMMARY OF THE INVENTION

Accordingly, in one aspect, the present invention provides a pharmaceutical combination for treating metabolic disorders, especially diabetes mellitus. Consumption of the combination of active drugs according to the invention affects the metabolic pathways of carbohydrate metabolism, resulting in less glucose getting into the body and more glucose in the bloodstream getting shunted to the muscles. Consumption of the combination according to the present invention by a subject promotes an increase in the ratio between lean and adipose tissue in the subject. In another aspect therefore, the combination is useful for achieving weight loss and weight control by preventing much of the calories of a carbohydrate-containing food from having an impact.

In yet another aspect, the present invention provides a method of treating diabetes and associated metabolic disorders comprising administration of Hydroxychloroquine and a DPP-IV inhibitor or their pharmaceutically acceptable salt thereof. The method comprises simultaneous, separate or sequential administration of effective amounts of the above active compounds and/or co-treatment, in a ratio which provides an additive and/or synergistic effect.

The present invention further provides a novel composition(s) or kit comprising of hydroxychloroquine or its pharmaceutically acceptable salts and DPP-IV inhibitors for simultaneous, separate, sequential, including in alternation, or combined use, especially in the prevention, delay of progression or treatment of conditions mediated by dipeptidylpeptidase-IV (DPP-IV), in particular diabetes, more particularly, type 2 diabetes mellitus, conditions of impaired glucose tolerance (IGT), conditions of impaired fasting plasma glucose, metabolic acidosis, ketosis, arthritis, obesity and osteoporosis, Specific DPP-4 inhibitors according to the invention are selected from, but not limited to, sitagliptin, vildagliptin, saxagliptin, melogliptin, P93/01 (Prosidion), alogliptin, denagliptin, Roche 0730699, TS021 (Taisho), E3024 (Eisai), linagliptin, carmegliptin, gosogliptin, teneligliptin and dutogliptin and their pharmaceutically acceptable salts.

### DETAILED DESCRIPTION OF THE PREFERRED EMBODIMENTS

The invention will now be described in detail in connection with certain preferred and optional embodiments, so that various aspects thereof may be more fully understood and appreciated. As used herein, the following terms and phrases shall have the meaning set forth below.

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described. To describe the invention, certain terms are defined herein specifically as follows.

Unless stated to the contrary, any of the words “including,” “includes,” “comprising,” and “comprises” mean “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

“Diabetes is art recognized term, and refers to high blood sugar or ketoacidosis, as well as chronic, general metabolic abnormalities arising from a prolonged high blood sugar status or a decrease in glucose tolerance. “Diabetes” encompasses both the Type 1 and Type 2 (Non Insulin Dependent Diabetes Mellitus or NIDDM) forms of the disease.

“Metabolic syndrome” is art recognized term, and the term stands for a group of risk factors that occur together and increase the risk for coronary artery disease, stroke, and type 2 diabetes.

The term “therapeutic effect” is art-recognized and refers to a local or systemic effect in animals, particularly mammals, and more particularly humans caused by a pharmacologically active substance. The phrase “therapeutically effective amount” means that amount of such a substance that produces some desired local or systemic effect at a reasonable benefit/risk ratio applicable to any treatment. The therapeutically effective amount of each substance will vary depending upon the subject, severity of disease or condition being treated, body weight and age of the subject, the manner of administration and the like, which can readily be determined by one or ordinary skill in the art. For example, certain compositions described herein may be administered in a sufficient amount to produce a desired effect at a reasonable benefit/risk ratio applicable to such treatment.

The present invention provides a method of delaying of progression in metabolic disorders comprising administration of Hydroxychloroquine along with a DPP-IV inhibitor or a pharmaceutically acceptable salt, solvate, or a prodrug thereof.

The invention provides a method for preventing, slowing the progression of, delaying, improving, restoring, or treating the diseases resulting from metabolic disorders comprising administration of Hydroxychloroquine along with a DPP-IV inhibitor or a pharmaceutically acceptable salt, solvate, or a prodrug thereof.

The invention provides a method for preventing, slowing the progression of, delaying, improving, restoring or treating a condition or a disorder selected from the group consisting of complications of diabetes such as abnormal physiological levels of biochemical parameters such as serum triglycerides, HDL cholesterol; cataracts, micro- and macrovascular diseases, such as nephropathy, retinopathy, neuropathy, tissue ischemia, diabetic foot, arteriosclerosis, myocardial infarction, acute coronary syndrome, unstable angina pectoris, stable angina pectoris, stroke, peripheral arterial occlusive disease, cardiomyopathy, heart failure, heart rhythm, disorders and vascular restenosis, or a combination thereof, in a patient in need thereof.

In another aspect, the method according to the present invention is useful for preventing, slowing, delaying the progression of, improving, restoring or treating the degeneration of pancreatic beta cells and/or the decline of the functionality of pancreatic beta cells, in a patient in need thereof.

In another aspect, the method according to the invention is useful for preventing, slowing, delaying the progression of, improving, restoring or treating diseases or conditions attributed to an abnormal accumulation of liver or ectopic fat, in a patient in need thereof.

In yet another aspect, the method according to the invention is useful for maintaining and/or improving the insulin sensitivity and/or for treating or preventing hyperinsulinemia and/or insulin resistance, in a patient in need thereof.

The invention further provides use of a pharmaceutical combination comprising hydroxychloroquine and a DPP-IV inhibitor in the manufacture of a medicament for preventing, slowing the progression of, delaying, improving, restoring, or treating the diseases resulting from metabolic disorders.

The present invention provides a method of preventing, slowing the progression of delaying, improving, restoring or treating diseases resulting from metabolic disorders such as diabetes comprising administration of Hydroxychloroquine along with a DPP-IV inhibitor or their pharmaceutically acceptable salt thereof. The method comprises simultaneous, separate, sequential, including in alternation, or combined administration of effective amounts of the above active compounds and/or co-treatment with other medications, in a ratio which provides an additive and/or synergistic effect. The data from the efficacy studies indicate that hydroxychloroquine potentiates the interaction of DPP IV inhibitor with their receptor(s) thereby resulting into synergistic impact on the lowering of blood glucose levels and obesity control.

The treatment aims diseases arising out of metabolic disorders, for example, those of (a) central obesity, indicated by excessive fat tissue in and around the abdomen; (b) dyslipidemia (blood fat disorders, mainly high triglycerides and low HDL cholesterol, that foster plaque buildups in artery walls); (c) elevated blood pressure; (d) insulin resistance or glucose intolerance (the body can't properly use insulin or blood sugar); (e) prothrombotic state (e.g., high fibrinogen or plasminogen activator inhibitor in the blood); (f) pro-inflammatory conditions such as osteoarthritis; and/or a combinations of one or more of the afore-mentioned diseases.

The method of treatment primarily useful for the control of blood glucose/sugar levels especially in Type 2 diabetes mellitus, apart from reduction of other bio-chemical parameters such as, HbA1c, and serum triglycerides. The combination not only reduces the level of bad cholesterol (LDL), but also increases the levels of good cholesterol (HDL).

Specific DPP-4 inhibitors according to the invention are selected from, but not limited to, sitagliptin, vildagliptin, saxagliptin, melogliptin, P93/01 (Prosidion), alogliptin, denagliptin, Roche 0730699, TS021 (Taisho), E3024 (Eisai), linagliptin, carmegliptin, gosogliptin, teneligliptin and dutogliptin and their pharmaceutically acceptable salts. One preferred DPP-4 inhibitor according to the invention is sitagliptin.

Accordingly, the preferred embodiment of the invention provides a pharmaceutical combination comprising hydroxychloroquine and a DPP-4 inhibitor, sitagliptin or a pharmaceutically acceptable salt, solvate, or a prodrug thereof.

The quantity of each substance to be administered will vary depending upon the DPP-IV inhibitor used, subject to be treated, severity of disease or condition being treated, body weight and age of the subject, the manner of administration and the like, which can readily be determined by one of ordinary skill in the art. The method for oral administration may comprise hydroxychloroquine, or its pharmaceutical salt for an average adult, in a quantity ranging from 50 mg to 500 mg calculated based on the weight of hydroxychloroquine free base, more preferably a dose of 150 to 400 mg may be administered as a daily dose. The DPP-IV inhibitor, may be administered in the range of 2 to 100 mg calculated based on the weight of DPP-IV inhibitor free base for example, Sitagliptin, as phosphate monohydrate salt for an average adult patient may be used in range of 5 to 100 mg per day, more preferably in an amount of 10 mg 50 mg.

Accordingly, a daily dosage of 100 mg to 500 mg of hydroxychloroquine free base and 3.5 mg to 80 mg of sitagliptin free base, or the corresponding dosage of the respective salt, solvate, or a prodrug thereof, may be conveniently administered to a patient in need thereof.

The pharmaceutical combination according to the present invention, wherein sitagliptin is fixed in a range from 3.5 mg to 80 mg calculated based on the weight of sitagliptin free base.

The pharmaceutical combination according to the present invention, wherein, sitagliptin is more preferably fixed in a range from 6 mg to 31 mg calculated based on the weight of sitagliptin free base.

The present combination of drugs or their pharmaceutically acceptable salts, solvate or prodrugs thereof can be delivered to the subject using a wide variety of routes or modes of administration. Suitable routes of administration include, but are not limited to, oral, transdermal, transmucosal, intestinal and parenteral administration, including intramuscular, subcutaneous and intravenous injections. A preferred mode of administration includes in oral dosage form.

Typically a fixed dose combination of hydroxychloroquine and DPP-IV inhibitor can be administered once or twice a day for short term or long term treatment to a patient suffering from metabolic syndrome/diseases, especially diabetes Mellitus. Long term treatment refers to an extended period of time, typically longer than two weeks, and includes any length of time whereby the individual/subject (mammal) exhibits improvement in disease conditions/symptoms. This dosage formulation will be beneficial for prophylactic treatment of individuals who are identified to develop metabolic disorders or pre-diabetic symptoms.

Prediabetes, also referred as borderline diabetes, impaired glucose tolerance (IGT), and/or impaired fasting glucose (IFG) is the state in which some but not all of the diagnostic criteria for diabetes are met. It is often described as the “gray area” between normal blood sugar and diabetic levels. While in this range, patients are at risk for not only developing type 2 diabetes, but also for cardiovascular complications. In a way, prediabetes is a misnomer since it is an early stage of diabetes and hence is recommended as a parameter to identify those who are at increased risk of developing the disease.

The present invention also provides novel composition(s) or kit or co-pack/combi-pack comprising of hydroxychloroquine or its pharmaceutically acceptable salts, solvates or prodrugs thereof and DPP-IV inhibitors for simultaneous, separate, sequential, including in alternation, or combined use, especially in the prevention, delay of progression or treatment of conditions mediated by dipeptidylpeptidase-IV (DPP-IV), and metabolic disorders, in particular diabetes, more particularly, type 2 diabetes mellitus, conditions of impaired glucose tolerance (IGT), conditions of impaired fasting plasma glucose, metabolic acidosis, ketosis, arthritis, obesity and osteoporosis.

The quantity of hydroxychloroquine or its pharmaceutically acceptable salts and DPP-IV inhibitors in the composition may be fixed in range from 100 mg to 500 mg calculated based on the weight of hydroxychloroquine free base, more preferably a dose of 150 to 400 mg and the DPP-IV inhibitor may be fixed in the range of 2 to 100 mg calculated based on the weight of DPP-IV inhibitor free base, for example, Sitagliptin, as phosphate monohydrate salt may be used in range of 5 to 100 mg, more preferably in an amount of 10 mg 50 mg, for oral use, respectively.

The composition of hydroxychloroquine along with DPP-IV inhibitors can be manufactured in combination with pharmaceutical carriers or diluents. For oral use, suitable pharmaceutical carriers include inert diluents or fillers thereby forming oral dosage forms such as tablets, powders, capsules, syrups or suspensions and the like.

The fixed dose formulation of the present invention is preferably in the form of tablets or capsules, wherein tablets/capsules can be prepared in immediate release, modified/controlled release, extended or in sustained release form. Dosage form may be, for example, but not limited to, a multilayer tablet, a two-layer tablet, or capsules or sachets containing the active ingredients in separate granulates or beads, either granulate or bead, optionally being coated with a protective coating or an enteric-coating.

For example, tablets containing variety of excipients such as disintegrants such as starch, complex silicates together with binding agents such as poly vinyl pyrrolidone, sucrose, gelatin and acacia. Lubricants such as magnesium silicate/stearate, sodium lauryl sulfate and talc are often used in tableting purposes. Solid compositions of the present invention can also be filled into soft and hard gelatin capsules.

For soft gelatin capsule, the composition may be solubilized in suitable vegetable or edible oil such as sunflower oil, corn oil, peanut oil or any other suitable oil.

The active ingredient (s) and excipients can be formulated into compositions and dosage forms according to methods known in the art.

The safety, efficacy and additive and synergistic effect of the combination of hydroxychloroquine with a DPP-IV inhibitor in type-2 diabetes is established by the following experiment.

### Pharmacological Evaluations:

In these studies we compared the efficacy in terms of potency of hydroxychloroquine sulphate with or without the DPP-IV inhibitor for example, Sitagliptin phosphate, Vildagliptin and Saxagliptin hydrochloride; and the biochemical parameters such as serum triglyceride, total cholesterol, SGOT, SGPT, creatinine and urea levels were monitored & compared with disease induced model & normal control rats.

### Study No 1

Study Design: Experimental Study on Fixed Dose Combination of Hydroxychloroquine with Sitagliptin

An experimental study was conducted in Institute of Pharmaceutical Research and Education, Wardha, India to demonstrate the efficacy of combination of hydroxychloroquine with sitagliptin in diabetes. In this experiment 48 Wistar rats weighing 130-150 g of either sex were divided randomly into 8 groups consisting of 6 rats each. The study drugs suspended in vehicle [0.1% w/v suspension of Tween 80 and carboxymethylcellulose (CMC) in water] were administered for orally for 21 days. Group 1 (normal vehicle control) consisted of normal rats that neither received streptozotocin nor any test drug; Group 2 served as streptozotocin induced diabetic control (Diabetes was induced in rats by single administration of streptozotocin (60 mg/kg/i.p) dissolved in 0.1 M-citrate buffer, pH 4.5). Forty-eight hours later, blood samples were collected and glucose levels were determined to confirm the development of diabetes. Only those animals which showed hyperglycemia (blood glucose levels>250 mg/dl) were considered as diabetic and received vehicle orally; Group 3 was diabetic rats treated with dose equivalent to human therapeutic dose of sitagliptin (STG) (11 mg/kg); Group 4 was diabetic rats treated with dose equivalent to human therapeutic dose of Hydroxychloroquine (HCQ) (46 mg/kg), Group 5 was diabetic rats treated with combination of HCQ 46 mg/kg and STG11 mg/kg; Group 6 was diabetic rats treated with subtherapeutic dose of HCQ 23 mg/kg; Group 7 was diabetic rats treated with subtherapeutic dose of STG 5.5 mg/kg and Group 8 was diabetic rats treated with combination of HCQ 23 mg/kg and STG 5.5 mg/kg.

Blood glucose level was measured in normal and experimental rats at 1^{st}, 7^{th}, 14^{th} and 21^{st} day of administration. For other biochemical parameters i.e. Insulin, HbA1c, Triglycerides, Total Cholesterol, HDL, SGOT, SGPT, Total Protein, Albumin, Creatinine and Urea Blood samples were collected 1 h after the last dose administration from 18 h fasted rats through the retro-orbital plexus under chloroform anesthesia and analyzed using autoanalyzer (Microlab 2000). Other parameters like body weight, food intake and water intake were evaluated weekly till day 21^{st}.

Baseline parameters did not differ significantly between the groups. After inducing diabetes, the serum levels of glucose, HbA1c, total cholesterol, triglycerides, SGOT, SGPT, creatinine and urea levels were high as compared to normal control rats. Oral administration of sitagliptin and hydroxychloroquine alone at therapeutic dose showed significant improvement in blood glucose levels on day 14^{th} while combination of STG and HCQ at therapeutic dose showed highly significant decrease in blood glucose on day 7^{th} as compared to diabetic control. Combination of therapeutic doses of hydroxychloroquine and sitagliptin showed significantly more fall in blood glucose levels as compared to individual treatment groups.

Serum Insulin levels were significantly reduced (p<0.01) in diabetic groups as compare to normal controls at 20 days (Table 2). Combination treatment of STG and HCQ for 21 days at therapeutic dose showed significant increase in serum insulin level as compared to diabetic control (p<0.01). Hb1Ac levels were significantly increased (p<0.01) in diabetic groups as compared to normal control at 21^{st} days (Table 2). Combination treatment of STG and HCQ for 21 days at therapeutic and subtherapeutic dose showed significant reduction in Hb1Ac level as compared to diabetic control (p<0.01).

Diabetic control rats showed significant rise in serum triglyceride, total cholesterol, SGOT, SGPT, creatinine and urea levels as compared to normal control rats. There were significant reductions in serum triglyceride, total cholesterol, SGOT, SGPT levels of the diabetic rats treated with combination of sitagliptin and hydroxychloroquine at therapeutic dose and sub-therapeutic dose as compared to the diabetic control group. Creatinine and serum urea level were significantly reduced only by combination treatment at therapeutic dose.

Similarly diabetic control rats showed significant fall in HDL-C, albumin & total protein levels as compared to normal control rats. After treatment for 21 days with therapeutic dose combinations there was significant rise in HDL-C levels as compared to the diabetic control group. While significant rise in albumin and total protein levels was observed in both the groups treated with therapeutic and sub-therapeutic dose combinations as compared to the diabetic control group. A significant reduction in body weight of diabetic animals was observed on day 14 and day 21 when compared with normal rats. Treatment of diabetic animals with combination shown significant increase in the body weight.

Average food and water intake in diabetic animals was increased significantly compared to normal animals. Combination treatment groups shown decrease in food and water intake.

TABLE 1
Effect of ST and HCQ on the blood glucose in STZ-induced diabetic rats

| Groups | | Plasma glucose concentration (mg/dl) | | | |
|---|---|---|---|---|---|
| (n = 6) | Treatment | 1^{st} day | 7^{th} day | 14^{th} day | 21^{st} day |
| I | Normal control | 92.33 ± 6.56 | 92.33 ± 7.03 | 92.17 ± 8.89 | 91.67 ± 5.65 |
| II | Diabetic control | 271.67 ± .91^{#} | 266.00 ± 14.70^{#} | 261.33 ± 14.88^{#} | 257.50 ± 14.10^{#} |
| III | STG (11 mg/kg) | 258.33 ± 11.57 | 225.00 ± 12.63* | 206.17 ± 10.26* | 156.50 ± 12.85* |
| IV | HCQ (46 mg/kg) | 267.83 ± 10.53 | 243.83 ± 10.78 | 230.00 ± 12.96* | 207.67 ± 7.53* |
| V | STG + HCQ(11 + 46 mg/kg)) | 255.33 ± 15.91 | 173.67 ± 10.46* | 133.50 ± 8.73* | 110.17 ± 8.77* |
| VI | STG (5.5 mg/kg) | 267.17 ± 12.83 | 261.83 ± 11.58 | 227.83 ± 13.95* | 200.83 ± 4.71* |
| VII | HCQ (23 mg/kg) | 253.33 ± 24.86 | 248.67 ± 24.63 | 241.33 ± 24.73 | 232.00 ± 19.92* |
| VIII | STG + HCQ(5.5 + | 261.83 ± 11.58* | 221.83 ± 23.25* | 197.17 ± 14.85* | 159.17 ± 10.42*^{∞} |
| | 23 mg/kg) | | | | |

Readings are mean values ± S.D. n = 6
^{#}p < 0.01 vs. Normal control.
*p < 0.01 vs. diabetic control..
STG: Sitagliptin;
HCQ: Hydroxychloroquine;

TABLE 2
Effect of ST and HCQ on the biochemical parameters in STZ-induced diabetic rats

| | | Biochemical Parameters | | | | | | | | | | |
|---|---|---|---|---|---|---|---|---|---|---|---|---|
| | | | | | | | | | Total | Albu- | | |
| Gr | Treatment | | | TG | TC | HDL-C | | | protein | min | Creati- | Urea |
| No | mg/kg/day | | HbA1c | (mg/dl) | (mg/dl) | (mg/dl) | SGOT | SGPT | (g/dl) | (g/dl) | nine | (mg/dl) |
| I. | Normal | 6.52 ± | 4.51 ± | 81.66 ± | 101.62 ± | 33.55 ± | 25.27 ± | 42.30 ± | 7.60 ± | 5.02 ± | 0.53 ± | 20.96 ± |
| | control | 0.48 | 0.24 | 4.75 | 4.80 | 1.64 | 1.47 | 1.20 | 0.44 | 0.21 | 0.06 | 0.1.32 |
| II | Diabetic | 4.67 ± | 6.22 ± | 131.08 ± | 171.00 ± | 25.18 ± | 57.86 ± | 56.98 ± | 4.05 ± | Insulin | 1.59 ± | 48.24 ± |
| | control | 1.14^{#} | 0.71^{#} | 12.46^{#} | 9.48^{#} | 2.66* | 2.24^{#} | 3.48^{#} | 0.51^{#} | | 0.35^{#} | 2.06^{#} |
| III | STG (11) | 6.11 ± | 5.04 ± | 116.48 ± | 146.92 ± | 26.04 ± | 51.47 ± | 55.47 ± | 4.83 ± | 2.63 ± | 1.63 ± | 23.57 ± |
| | | 0.76** | 0.54* | 6.71** | 6.79** | 2.14 | 1.28** | 1.93* | 0.31* | 0.37 | 0.23 | 1.49** |
| IV | HCQ (46) | 5.97 ± | 4.91 ± | 124.68 ± | 158.57 ± | 25.07 ± | 53.77 ± | 55.94 ± | 4.12 ± | 2.33 ± | 1.72 ± | 43.85 ± |
| | | 1.10 | 0.88** | 5.94 | 9.36* | 2.06 | 1.91** | 2.15 | 0.36 | 0.25 | 0.25 | 2.22** |
| V | STG + HCQ | 7.08 ± | 4.61 ± | 89.22 ± | 114.67 ± | 31.97 ± | 32.28 ± | 45.10 ± | 6.18 ± | 4.35 ± | 0.67 ± | 26.55 ± |
| | (11 + 46) | 1.06** | 0.41** | 6.23** | 5.25** | 0.93** | 1.32** | 1.54** | 0.39** | 0.29** | 0.05** | 1.56** |
| VI | STG (5.5) | 4.43 ± | 5.12 ± | 121.91 ± | 156.69 ± | 23.92 ± | 54.02 ± | 54.64 ± | 4.15 ± | 2.20 ± | 1.76 ± | 45.05 ± |
| | | 1.46 | 0.97* | 5.70 | 4.13** | 2.08 | 1.01** | 1.47** | 0.59 | 0.25 | 0.07 | 2.14* |
| VII | HCQ (23) | 4.71 ± | 5.30 ± | 130.65 ± | 166.69 ± | 24.99 ± | 57.29 ± | 58.12 ± | 4.03 ± | 2 23 ± | 1.79 ± | 46.38 ± |
| | | 1.06 | 0.64 | 4.37 | 4.05 | 1.81 | 1.16 | 1.93 | 0.26 | 0.29 | 0.16 | 2.99 |
| VII | STG + HCQ | 5.15 ± | 4.61 ± | 103.62 ± | 135.80 ± | 27.32 ± | 45.79 ± | 52.73 ± | 5.20 ± | 2.97 ± | 1.32 ± | 42.41 ± |
| | (5.5 + 23) | 1.07 | 0.34** | 4.85** | 5.19** | 0.99 | 1.66** | 1.61** | 0.32** | 0.32** | 0.15 | 1.14** |

Readings are values ± S.E. n = no. of animals in each group.
^{#}p < 0.01 vs. Normal control.
*p < 0.05,
**p < 0.01 vs. diabetic control.).
STG: Sitagliptin; HCQ: Hydroxychloroquine;

TABLE 3
Effect of ST and HCQ on Body weight in STZ-induced diabetic rats

| | Body weight (g) | | | |
|---|---|---|---|---|
| Groups | Day 1 | Day 7 | Day 14 | Day 21 |
| Normal | 149.17 ± 16.98 | 155.83 ± 19.45 | 172.67 ± 20.31 | 175.33 ± 20.91 |
| control | | | | |
| Diabetic | 146.00 ± 8.22^{#} | 142.17 ± 7.81^{#} | 133.17 ± 6.77^{#} | 122.00 ± 4.34^{#} |
| control | | | | |
| STG | 137.83 ± 6.49 | 142.67 ± 7.45 | 149.00 ± 7.46 | 154.50** ± 6.92 |
| (11 mg/kg) | | | | |
| HCQ | 140.33 ± 14.60 | 141.17 ± 14.26 | 142.67 ± 13.52 | 143.50* ± 14.10 |
| (46 mg/kg) | | | | |
| STG + HCQ | 136.67 ± 9.77 | 144.50 ± 7.71 | 153.00 ± 9.27 | 160.83** ± 9.64 |
| (11 + 46 mg/kg) | | | | |
| STG (5.5 mgkg) | 145.83 ± 12.61 | 147.83 ± 12.37 | 150.50 ± 12.31 | 152.33** ± 11.66 |
| HCQ (46 mg/kg) | 133.33 ± 17.67 | 133.67 ± 16.61 | 135.17 ± 16.29 | 137.00 ± 15.75 |
| STG + HCQ | 128.50 ± 14.54 | 134.17 ± 14.69 | 139.50 ± 14.47 | 145.67 ± 14.22* |
| (5.5 + 23 mg/kg) | | | | |

Readings are values ± S.E. n = no. of animals in each group.
^{#}p < 0.01 vs. Normal control.
*p < 0.05,
**p < 0.01 vs. diabetic control.).
STG: Sitagliptin;
HCQ: Hydroxychloroquine;

TABLE 4
Effect of ST and HCQ on Food intake in STZ-induced diabetic rats

AVERAGE FOOD INTAKE (g)

| Groups | Day 1 | Day 7 | Day 14 | Day 21 |
|---|---|---|---|---|
| Normal | 9.50 | 8.83 | 8.50 | 8.33 |
| Diabetic | 11.17 | 12.17 | 13.00 | 14.00 |
| HCQ (46 mg/kg) | 11.50 | 10.33 | 8.67 | 8.33 |
| STG (11 mg/kg) | 12.17 | 10.67 | 9.83 | 8.83 |
| HCQ + STG | 13.17 | 11.33 | 9.50 | 8.17 |
| (11 + 46 mg/kg) | | | | |
| HCQ (23 mg/kg) | 12.17 | 11.50 | 11.83 | 11.67 |
| STG (5.5 mg/kg) | 11.50 | 11.83 | 10.83 | 10.33 |
| HCQ + STG | 11.83 | 10.50 | 9.50 | 8.50 |
| (5.5 + 23 mg/kg) | | | | |

TABLE 5
Effect of ST and HCQ on Water intake in STZ-induced diabetic rats

AVERAGE WATER INTAKE (ml)

| Groups | Day 1 | Day 7 | Day 14 | Day 21 |
|---|---|---|---|---|
| Normal | 27.50 | 29.67 | 26.33 | 27.50 |
| Diabetic | 35.00 | 38.33 | 44.67 | 48.33 |
| HCQ The | 30.00 | 29.17 | 28.33 | 28.83 |
| STG The | 30.67 | 29.00 | 28.50 | 27.17 |
| HCQ + STG The | 25.00 | 23.67 | 23.00 | 22.00 |
| HCQ Sub-The | 31.67 | 32.17 | 32.17 | 31.83 |
| STG Sub-The | 28.33 | 27.17 | 26.67 | 27.00 |
| HCQ + STG Sub- | 32.50 | 30.33 | 29.67 | 30.00 |
| The | | | | |

The results shows marked reduction in blood glucose levels, total cholesterol, reduction in LDL, triglycerides, normalization of SGOT & SGPT, and rise in HDL; which clearly demonstrates for the first time that the combination therapy of hydroxychloroquine with DPP-IV inhibitor such as sitagliptin has a greater antihyperglycemic effect with normalization of other metabolic disease symptoms compared to their monotherapy.

### Study No 2

Experimental Study on Fixed Dose Combination of Hydroxychloroquine with Vildagliptin and Saxagliptin

An experimental study was conducted at Institute of Pharmaceutical Research and Education, Wardha, India to demonstrate the efficacy of combination of hydroxychloroquine with vildagliptin and saxagliptin in diabetes. In this experiment 96 Wistar rats weighing 150-200 g of either sex were divided randomly into 16 groups consisting of 6 rats each. The study drugs were dissolved in water/suspended in vehicle [0.1% w/v suspension of Tween 80 and carboxymethylcellulose (CMC) in water] and administered orally for 28 days. Group 1 (normal vehicle control) consisted of normal rats that neither received Streptozotocin (STZ) nor any test drug; Group 2 served as Streptozotocin induced diabetic control [Diabetes was induced in rats by single administration of Streptozotocin (55 mg/kg/i.p.) dissolved in 0.1 M-citrate buffer, pH 4.5]. Forty-eight hours later, blood samples were collected and glucose levels were determined to confirm the development of diabetes. Only animals showing hyperglycaemia (blood glucose levels>250 mg/dl) were considered as diabetic and received vehicle orally; Group 3 was diabetic rats treated with hydroxychloroquine (HCQ) (22.5 mg/kg); Group 4 was diabetic rats treated with HCQ (45 mg/kg), Group 5 was diabetic rats treated with vildagliptin (VG) 6 mg/kg; Group 6 was diabetic rats treated with VG 12 mg/kg; Group 7 was diabetic rats treated with HCQ 22.5 mg/kg+VG 12 mg/kg and Group 8 was diabetic rats treated with combination of HCQ 45 mg/kg and VG 6 mg/kg; Group 9 was diabetic rats treated with saxagliptin (SG) 0.3 mg/kg; Group 10 was diabetic rats treated with Saxagliptin (SG) 0.6 mg/kg; Group 11 was diabetic rats treated with combination of HCQ 45 mg/kg+SG 0.3 mg/kg; Group 12 was diabetic rats treated with combination of HCQ 22.5 mg/kg+SG 0.6 mg/kg; Group 13 was diabetic rats treated with combination of HCQ 22.5 mg/kg and VG 6 mg/kg; Group 14 was diabetic rats treated with combination of HCQ 45 mg/kg and VG 12 mg/kg; Group 15 was diabetic rats treated with combination of HCQ 22.5 mg/kg+SG 0.3 mg/kg; Group 16 was diabetic rats treated with combination of HCQ 45 mg/kg+SG 0.6 mg/kg.

Blood glucose level was measured in normal and experimental rats at 0, 7^{th}, 14^{th}, 21^{st} and 28^{th} day of administration. For other biochemical parameters i.e. HbA1c, Total Protein, Creatinine and Urea blood samples were collected 1 h after the last dose administration from 18 h fasted rats through the retro-orbital plexus under chloroform anaesthesia and analyzed using autoanalyzer. Other parameters like body weight, food intake and water intake were evaluated weekly till day 28.

After inducing diabetes, the serum levels of glucose, HbA1c, urea and creatinine levels were high as compared to normal control rats. Oral administration of HCQ, VG, and SG alone at therapeutic equivalent doses showed significant improvement in blood glucose levels on day 28 as compared to diabetic control group. Combination of HCQ (45 mg/kg)+VG (6 mg/kg); HCQ (22.5 mg/kg)+VG (12 mg/kg); HCQ (45 mg/kg)+VG (12 mg/kg) showed significantly more fall in blood glucose levels as compared to their individual treatment groups. Also the combination of HCQ with SG at all the dose levels tested showed significantly more fall in blood glucose levels as compared to their individual treatment groups (Table 6).

TABLE 6
Effect of HCQ, SG and VG on the average blood glucose in STZ induced diabetic rats

| Group | | Plasma glucose concentration (mg/dl) | | | | |
|---|---|---|---|---|---|---|
| (n = 6) | Treatment | Day 0 | Day 7 | Day 14 | Day 21 | Day 28 |
| 1 | Normal | 84.25 ± 4.41 | 84.76 ± 4.11 | 85.91 ± 4.61 | 89.72 ± 5.38 | 89.9 ± 11.22 |
| 2 | Diabetic | 301.17 ± 33.69 | 303.83 ± 24.97 | 293.33 ± 15.9 | 288.83 ± 19.95 | 283.33 ± 21.3 |
| 3 | HCQ | 292.17 ± 26.12 | 278 ± 22.59 | 269.33 ± 11.45 | 266.67 ± 24.68 | 266.5 ± 11.33 |
| | (22.5 mg/kg) | | | | | |
| 4 | HCQ | 305.67 ± 27.14 | 265.5 ± 8.87 | 266.5 ± 15.86 | 265.83 ± 9.22 | 244.5 ± 18.1* |
| | (45 mg/kg) | | | | | |
| 5 | VG (6 mg/kg) | 296 ± 21.59 | 242.17 ± 40.58 | 199.83 ± 35.33 | 191.33 ± 22.46 | 182.67 ± 33.01* |
| 6 | VG (12 mg/kg) | 288 ± 11.58 | 227 ± 40.57 | 188.83 ± 34.23 | 173.5 ± 15.6 | 167.5 ± 9.12* |
| 7 | HCQ + VG | 286 ± 21.91 | 146.5 ± 4.89 | 144 ± 8.67 | 143.5 ± 13.38 | 138.33 ± 17.9** |
| | (22.5 + 12 mg/kg) | | | | | |
| 8 | HCQ + VG | 297.67 ± 32.41 | 162.33 ± 6.71 | 158.33 ± 7.03 | 155.67 ± 7.97 | 145.17 ± 13.33** |
| | (45 + 6 mg/kg) | | | | | |
| 9 | SG (0.3 mg/kg) | 288.83 ± 26.19 | 246.5 ± 4.89 | 226.33 ± 12.36 | 194 ± 38.79 | 194.17 ± 17.17* |
| 10 | SG (0.6 mg/kg) | 285.5 ± 33.58 | 146.5 ± 4.89 | 138.5 ± 7.23 | 137.5 ± 9.44 | 161 ± 9.67* |
| 11 | HCQ + SG | 283.33 ± 21.3 | 246.83 ± 9.62 | 202 ± 43.29 | 194.5 ± 32.72 | 167.17 ± 17.53## |
| | (45 + 0.3 mg/kg) | | | | | |
| 12 | HCQ + SG | 297.67 ± 25.9 | 143.67 ± 16.37 | 141.17 ± 12.45 | 138.33 ± 9.16 | 134.67 ± 11.76## |
| | (22.5 + 0.6 mg/kg) | | | | | |
| 13 | HCQ + VG | 286.5 ± 20.06 | 250.33 ± 26.03 | 229.33 ± 24.39 | 194 ± 12.38 | 177.5 ± 10.11 |
| | (22.5 + 6 mg/kg) | | | | | |
| 14 | HCQ + VG | 294.67 ± 7.87 | 255.33 ± 18.9 | 203 ± 15.77 | 174.17 ± 11.23 | 143.5 ± 7.26** |
| | (45 + 12 mg/kg) | | | | | |
| 15 | HCQ + SG | 292.17 ± 18.15 | 265.33 ± 18.34 | 232.83 ± 22.56 | 190.5 ± 13.1 | 158.83 ± 12.51## |
| | (22.5 + 0.3 mg/kg) | | | | | |
| 16 | HCQ + SG | 285.83 ± 16.46 | 248.17 ± 19 | 200.17 ± 17.77 | 171.67 ± 6.89 | 137.5 ± 8.36## |
| | (45 + 0.6 mg/kg) | | | | | |

HCQ: Hydroxychloroquine; VG: Vildagliptin; SG: Saxagliptin
*indicates p-value ≦ 0.05 vs. Diabetic control.
**indicates p-value ≦ 0.05 vs. respective doses of VG and HCQ monotherapies
##indicates p-value ≦ 0.05 vs. respective doses of SG and HCQ monotherapies

HbA1c levels were significantly increased (p≦0.05) in diabetic groups as compared to normal control group at 28 days (Table 7). Treatment with the monotherapies of HCQ, VG and SG significantly (p≦0.05) reduced the HbA1c levels as compared to diabetic control group at all tested doses. Combination treatment of HCQ+VG and HCQ+SG at all tested dose levels for 28 days showed significant reduction in HblAc level as compared to diabetic control (p≦0.05) (Table 7). Moreover the combination of HCQ 22.5 mg/kg+VG 6 mg/kg and HCQ 45 mg/kg+VG 6 mg/kg (P≦0.05) reduced the HbA1c levels significantly as compared to monotherapy with VG 6 mg/kg. Further to this when combination of HCQ 22.5 mg/kg+VG 12 mg/kg and HCQ 45 mg/kg+VG 12 mg/kg was administered more reduction was observed in HbA1c levels as compared to monotherapy with VG 12 mg/kg. Similar result was observed with the combination of HCQ 22.5 mg/kg+SG 0.3 mg/kg and HCQ 45 mg/kg+SG 0.3 mg/kg (P≦0.05) reduced the HbA1c levels more significantly as compared to monotherapy with SG 0.3 mg/kg. When combination of HCQ22.5 mg/kg+SG0.6 mg/kg and HCQ 45 mg/kg+SG 0.6 mg/kg was administered more reduction was observed in HbA1c levels as compared to monotherapy with SG 0.6 mg/kg (Table 7).

TABLE 7
Effect of HCQ, VG and SG on the biochemical parameters in STZ-induced
diabetic rats

| | | Biochemical parameters | | | |
|---|---|---|---|---|---|
| | | | | Total | |
| Gr. | Treatment | | | Protein | Creatinine |
| No. | (mg/kg/day) | HbA1c (%) | Urea (mg/dl) | (gm/dl) | (mg/dL) |
| 1 | Normal | 5.90 ± 0.41 | 18.81 ± 0.84 | 8.74 ± 0.17 | 0.88 ± 0.08 |
| 2 | Diabetic | 12.57 ± 0.92 | 28.22 ± 1.25 | 5.40 ± 0.45 | 2.26 ± 0.32 |
| 3 | HCQ (22.5 mg/kg) | 11.28 ± 1.08* | 24.68 ± 1.96* | 6.16 ± 0.41* | 1.84 ± 0.46 |
| 4 | HCQ (45 mg/kg) | 11.07 ± 1.22* | 23.37 ± 2.86* | 6.35 ± 0.79* | 1.75 ± 0.29* |
| 5 | VG (6 mg/kg) | 10.76 ± 0.97* | 21.48 ± 4.35* | 7.08 ± 0.55* | 1.78 ± 0.14* |
| 6 | VG (12 mg/kg) | 9.97 ± 0.95* | 20.23 ± 1.89* | 7.15 ± 0.19* | 1.69 ± 0.21* |
| 7 | HCQ + VG | 8.50 ± 0.70** | 19.84 ± 0.54 | 6.11 ± 0.59** | 1.65 ± 0.19 |
| | (22.5 + 12 mg/kg) | | | | |
| 8 | (HCQ + VG | 8.76 ± 1.68** | 20.29 ± 2.08 | 6.20 ± 0.52** | 1.71 ± 0.14 |
| | (45 + 6 mg/kg) | | | | |
| 9 | SG (0.3 mg/kg) | 10.79 ± 1.05* | 22.74 ± 2.71* | 6.55 ± 0.37* | 1.89 ± 0.23* |
| 10 | SG (0.6 mg/kg) | 9.65 ± 1.51* | 21.26 ± 2.41* | 6.62 ± 0.49* | 1.81 ± 0.27* |
| 11 | HCQ + SG | 9.08 ± 1.23^{##} | 23.54 ± 1.51 | 7.89 ± 0.95^{##} | 0.76 ± 0.26 |
| | (45 + 0.3 mg/kg) | | | | |
| 12 | HCQ + SG | 8.33 ± 0.83^{##} | 19.97 ± 2.04 | 7.00 ± 0.62 | 1.76 ± 0.23 |
| | (22.5 + 0.6 mg/kg) | | | | |
| 13 | HCQ + VG | 9.56 ± 0.71** | 20.86 ± 1.89 | 7.76 ± 0.47** | 1.73 ± 0.18 |
| | (22.5 + 6 mg/kg) | | | | |
| 14 | HCQ + VG | 8.09 ± 1.11** | 19.14 ± 1.81 | 7.99 ± 0.51** | 1.59 ± 0.23 |
| | (45 + 12 mg/kg) | | | | |
| 15 | HCQ + SG | 9.20 ± 0.81^{##} | 21.11 ± 3.17 | 7.42 ± 0.89^{##} | 1.78 ± 0.22 |
| | (22.5 + 0.3 mg/kg) | | | | |
| 16 | HCQ + SG | 7.86 ± 1.51^{##} | 18.25 ± 1.23^{##} | 7.38 ± 0.49^{##} | 1.67 ± 0.13 |
| | (45 + 0.6 mg/kg) | | | | |

HCQ: Hydroxychloroquine;
VG: Vildagliptin;
SG: Saxagliptin
*indicates p-value ≦0.05 vs. Diabetic control.
**indicates p-value ≦0.05 vs. respective doses of VG and HCQ monotherapies
^{##}indicates p-value ≦0.05 vs. respective doses of SG and HCQ monotherapies
HCQ: Hydroxychloroquine;
VG: Vildagliptin;
SG: Saxagliptin

Diabetic control rats showed rise in creatinine and urea levels as compared to normal control rats. Administration of monotherapies of HCQ, VG and SG showed significant reduction in urea and creatinine levels as compared to diabetic control group (p≦0.05) at all the tested dose levels except in HCQ (22.5 mg/kg) group. Although not significant HCQ 22.5 mg/kg monotherapy also showed reduction in creatinine level as compared to diabetic control group (Table 7) Similarly combination of HCQ with VG in all tested doses showed more reduction in urea and creatinine levels as compared to their respective monotherapies (Table 7). Similar reducing trend was observed in urea and creatinine levels after administration of combination of HCQ with SG at all tested doses. Moreover the reduction in urea level by combination of HCQ 45 mg/kg+SG 0.6 mg/kg was more significant as compared to SG 0.6 mg/kg (p≦0.05). Diabetic control rats showed fall in total protein levels as compared to normal control rats. After treatment for 28 days with the monotherapies of HCQ, VG and SG at all doses showed significant rise in total protein level as compared to the diabetic control group (p≦0.05) (Table 7). Moreover the increase in the total protein levels was significantly more in group treated with combination of HCQ+VG and HCQ+SG at all doses except HCQ 22.5 mg/kg+SG 0.6 mg/kg compared to their respective monotherapies. A significant reduction in body weight of diabetic animals was observed on day 28 when compared with normal rats. Treatment of diabetic animals with monotherapies of HCQ showed significant increase in the body weight (p≦0.05) (Table 8). In animals treated with monotherapies of VG and SG the body weights were reduced. However this reduction was significantly less as compared to diabetic control group (p≦0.05). However the reduction in body weight in groups treated with combination of HCQ with VG and HCQ with SG was comparable with monotherapies of VG and SG. Average food and water intake in diabetic animals was increased compared to normal animals. Combination treatment groups showed decreasing trend in food intake (Table 10) and water intake (Table 9).

TABLE 8
Effect of HCQ, VG and SG on body weight in STZ-induced diabetic rats

| Gr. | Treatment | Body Weight (g) | | | | |
|---|---|---|---|---|---|---|
| No. | (mg/kg/day) | Day 0 | Day 7 | Day 14 | Day 21 | Day 28 |
| 1 | Normal | 202.83 ± 83.3 | 207.83 ± 8.68 | 215.5 ± 7.34 | 224.67 ± 6.28 | 229 ± 8.34 |
| 2 | Diabetic | 200.67 ± 11.91 | 182.33 ± 6.06 | 177.83 ± 5.71 | 171.83 ± 6.85 | 166.33 ± 8.71 |
| 3 | HCQ | 199.83 ± 17.7 | 190.67 ± 11.41 | 196.17 ± 10.93 | 199.67 ± 10.52 | 201.17 ± 8.7* |
| | (22.5 mg/kg) | | | | | |
| 4 | HCQ | 198 ± 11.03 | 184.17 ± 9.99 | 190.67 ± 9.56 | 194.67 ± 9.52 | 198.17 ± 9.15* |
| | (45 mg/kg) | | | | | |
| 5 | VG (6 mg/kg) | 195 ± 7.18 | 179.17 ± 11.27 | 182.83 ± 12.66 | 185.67 ± 11.86 | 189.5 ± 11.81* |
| 6 | VG (12 mg/kg) | 197.5 ± 6.35 | 189 ± 3.52 | 187.67 ± 6.38 | 186.5 ± 8.83 | 185.67 ± 11.69* |
| 7 | HCQ + VG | 200.5 ± 6.22 | 192.83 ± 8.26 | 191.17 ± 7.81 | 188.17 ± 8.26 | 186.17 ± 8.28 |
| | (22.5 + 12 mg/kg) | | | | | |
| 8 | HCQ + VG | 206.17 ± 5.6 | 198.67 ± 5.99 | 197 ± 5.93 | 195.5 ± 6.16 | 194.17 ± 5.81 |
| | (45 + 6 mg/kg) | | | | | |
| 9 | SG (0.3 mg/kg) | 208.33 ± 6.15 | 199.5 ± 7.69 | 197.83 ± 8.06 | 196.33 ± 8.36 | 193.83 ± 8.13* |
| 10 | SG (0.6 mg/kg) | 211 ± 7.38 | 203.17 ± 7.47 | 201.17 ± 8.52 | 199.83 ± 8.35 | 198 ± 8.74* |
| 11 | HCQ + SG | 212.83 ± 7.44 | 203.5 ± 8.43 | 201.67 ± 7.12 | 200.33 ± 6.89 | 198.67 ± 6.98 |
| | (45 + 0.3 mg/kg) | | | | | |
| 12 | HCQ + SG | 215.17 ± 11.62 | 212.83 ± 11.16 | 210.5 ± 12.32 | 208.67 ± 12.48 | 206.83 ± 13.08 |
| | (22.5 + 0.6 mg/kg) | | | | | |
| 13 | HCQ + VG | 202.5 ± 6.57 | 192.67 ± 4.18 | 190.17 ± 4.79 | 189.17 ± 5.38 | 188.83 ± 4.83 |
| | (22.5 + 6 mg/kg) | | | | | |
| 14 | HCQ + VG | 200.67 ± 10.46 | 189 ± 7.67 | 186.83 ± 5.42 | 189 ± 5.69 | 193.17 ± 5.56 |
| | (45 + 12 mg/kg) | | | | | |
| 15 | HCQ + SG | 201.5 ± 9.14 | 191.83 ± 3.76 | 190.83 ± 3.49 | 191.17 ± 3.25 | 194.5 ± 6.44 |
| | (22.5 + 0.3 mg/kg) | | | | | |
| 16 | HCQ + SG | 199 ± 6.93 | 189.83 ± 10.01 | 190.33 ± 7.53 | 189.83 ± 8.33 | 193.5 ± 6.47 |
| | (45 + 0.6 mg/kg) | | | | | |

HCQ: Hydroxychloroquine; VG: Vildagliptin; SG: Saxagliptin
*indicates p-value ≦ 0.05 vs. Diabetic control.
**indicates p-value ≦ 0.05 vs. respective doses of VG and HCQ monotherapies
^{##} indicates p-value ≦ 0.05 vs. respective doses of SG and HCQ monotherapies

TABLE 9
Effect of HCQ, VG and SG on average water
intake in STZ induced diabetic rats

| | | AVERAGE WATER INTAKE (ml) | | | | |
|---|---|---|---|---|---|---|
| Gr. | Treatment | Day | Day | Day | Day | Day |
| No. | (mg/kg/day) | 0 | 7 | 14 | 21 | 28 |
| 1 | Normal | 23.00 | 20.56 | 20.05 | 23.33 | 24.56 |
| 2 | Diabetic | 25.25 | 23.99 | 22.50 | 27.00 | 26.11 |
| 3 | HCQ | 25.05 | 24.11 | 23.29 | 26.34 | 25.44 |
| | (22.5 mg/kg) | | | | | |
| 4 | HCQ | 23.88 | 22.41 | 21.55 | 26.22 | 25.79 |
| | (45 mg/kg) | | | | | |
| 5 | VG (6 mg/kg) | 25.00 | 22.12 | 21.32 | 25.12 | 26.31 |
| 6 | VG (12 mg/kg) | 24.35 | 22.19 | 22.11 | 26.13 | 25.71 |
| 7 | HCQ + VG | 24.16 | 22.36 | 21.61 | 25.72 | 26.00 |
| | (22.5 + 12 mg/kg) | | | | | |
| 8 | HCQ + VG | 22.11 | 22.36 | 21.55 | 25.57 | 25.00 |
| | (45 + 6 mg/kg) | | | | | |
| 9 | SG (0.3 mg/kg) | 23.71 | 21.12 | 21.67 | 25.17 | 25.19 |
| 10 | SG (0.6 mg/kg) | 24.86 | 21.11 | 21.89 | 26.00 | 25.12 |
| 11 | HCQ + SG | 24.66 | 22.13 | 21.99 | 26.16 | 25.87 |
| | (45 + 0.3 mg/kg) | | | | | |
| 12 | HCQ + SG | 24.15 | 22.51 | 22 | 25.00 | 26.12 |
| | (22.5 + 0.6 mg/kg) | | | | | |
| 13 | HCQ + VG | 23.16 | 26.00 | 24.16 | 25.26. | 25.46 |
| | (22.5 + 6 mg/kg) | | | | | |
| 14 | HCQ + VG | 25.42 | 21.85 | 28.16 | 26.00 | 22.31 |
| | (45 + 12 mg/kg) | | | | | |
| 15 | HCQ + SG | 20.63 | 21.46 | 24.63 | 27.16 | 20.16 |
| | (22.5 + 0.3 mg/kg) | | | | | |
| 16 | HCQ + SG | 23.48 | 22.36 | 26.45 | 22.23 | 27.46 |
| | (45 + 0.6 mg/kg) | | | | | |

HCQ: Hydroxychloroquine; VG: Vildagliptin; SG: Saxagliptin

TABLE 10
Effect of HCQ, VG and SG on food intake
in STZ induced diabetic rats

| | | AVERAGE FOOD INTAKE (g) | | | | |
|---|---|---|---|---|---|---|
| Gr. | Treatment | Day | Day | Day | Day | Day |
| No. | (mg/kg/day) | 0 | 7 | 14 | 21 | 28 |
| 1 | Normal | 11.11 | 13.23 | 12.13 | 12.00 | 14.35 |
| 2 | Diabetic | 10.98 | 12.75 | 11.90 | 12.00 | 13.89 |
| 3 | HCQ | 11.45 | 13.35 | 11.95 | 13.00 | 14.11 |
| | (22.5 mg/kg) | | | | | |
| 4 | HCQ | 11.67 | 13.39 | 12.05 | 12.75 | 14.00 |
| | (45 mg/kg) | | | | | |
| 5 | VG (6 mg/kg) | 11.40 | 13.00 | 12.10 | 12.90 | 14.33 |
| 6 | VG (12 mg/kg) | 11.90 | 13.50 | 12.00 | 13.05 | 14.30 |
| 7 | HCQ + VG | 11.00 | 13.05 | 12.50 | 13.00 | 14.50 |
| | (22.5 + 12 mg/kg) | | | | | |
| 8 | HCQ + VG | 12.00 | 14.10 | 12.76 | 12.90 | 14.32 |
| | (45 + 6 mg/kg) | | | | | |
| 9 | SG (0.3 mg/kg) | 12.13 | 14.00 | 12.54 | 13.05 | 14.25 |
| 10 | SG (0.6 mg/kg) | 12.45 | 14.45 | 12.00 | 13.26 | 14.00 |
| 11 | HCQ + SG | 12.07 | 14.06 | 12.36 | 13.00 | 13.86 |
| | (45 + 0.3 mg/kg) | | | | | |
| 12 | HCQ + SG | 12.50 | 14.00 | 12.54 | 13.57 | 13.55 |
| | (22.5 + 0.6 mg/kg) | | | | | |
| 13 | HCQ + VG | 12.00 | 11.50 | 13.50 | 13.00 | 13.50 |
| | (22.5 + 6 mg/kg) | | | | | |
| 14 | HCQ + VG | 12.50 | 12.00 | 12.50 | 12.50 | 12.50 |
| | (45 + 12 mg/kg) | | | | | |
| 15 | HCQ + SG | 13.25 | 13.50 | 14.50 | 14.50 | 12.50 |
| | (22.5 + 0.3 mg/kg) | | | | | |
| 16 | HCQ + SG | 12.50 | 12.00 | 13.00 | 13.50 | 13.00 |
| | (45 + 0.6 mg/kg) | | | | | |

HCQ: Hydroxychloroquine; VG: Vildagliptin; SG: Saxagliptin

The following examples, which include preferred embodiments, will serve to illustrate the practice of this invention, it being understood that the particulars shown are by way of example and for purpose of illustrative discussion of preferred embodiments of the invention.

### Example 1

i. Each tablet/capsule contains:

| | |
|---|---|
| Hydroxychloroquine | 100 mg |
| Sitagliptin | 25 mg/50 mg |
| Excipients q.s. | to make 200 mg tablet/capsule |

ii. Each tablet/capsule contains:

| | |
|---|---|
| Hydroxychloroquine | 200 mg |
| Sitagliptin | 25 mg/50 mg |
| Excipients q.s. | to make 400 mg tablet/capsule |

iii. Each tablet/capsule contains:

| | |
|---|---|
| Hydroxychloroquine | 300 mg |
| Sitagliptin | 50 mg/100 mg |
| Excipients q.s. | to make 500 mg tablet/capsule |

iv. Each tablet/capsule contains:

| | |
|---|---|
| Hydroxychloroquine | 400 mg |
| Sitagliptin | 25 mg/50 mg |
| Excipients q.s. | to make 600 mg tablet/capsule |

### Example 3

i. Each tablet/capsule contains:

| | |
|---|---|
| Hydroxychloroquine | 100 mg |
| Vildagliptin | 25 mg/50 mg |
| Excipients q.s. | to make 200 mg tablet/capsule |

ii. Each tablet/capsule contains:

| | |
|---|---|
| Hydroxychloroquine | 200 mg |
| Vildagliptin | 25 mg/50 mg |
| Excipients q.s. | to make 300 mg tablet/capsule |

iii. Each tablet/capsule contains:

| | |
|---|---|
| Hydroxychloroquine | 300 mg |
| Vildagliptin | 100 mg |
| Excipients q.s. | to make 400 mg tablet/capsule |

iv. Each tablet/capsule contains:

| | |
|---|---|
| Hydroxychloroquine | 400 mg |
| Vildagliptin | 50 mg |
| Excipients q.s. | to make 600 mg tablet/capsule |

### Example 4

i. Each tablet/capsule contains:

| | |
|---|---|
| Hydroxychloroquine | 100 mg |
| Saxagliptin | 2.5 mg/5 mg |
| Excipients q.s. | to make 200 mg tablet/capsule |

ii. Each tablet/capsule contains:

| | |
|---|---|
| Hydroxychloroquine | 200 mg |
| Saxagliptin | 2.5 mg/5 mg |
| Excipients q.s. | to make 300 mg tablet/capsule |

iii. Each tablet/capsule contains:

| | |
|---|---|
| Hydroxychloroquine | 300 mg |
| Saxagliptin | 2.5 mg/5 mg |
| Excipients q.s. | to make 400 mg tablet/capsule |

iv. Each tablet/capsule contains:

| | |
|---|---|
| Hydroxychloroquine | 400 mg |
| Saxagliptin | 2.5 mg/5 mg |
| Excipients q.s. | to make 500 mg tablet/capsule |

### Example 5

i. Each tablet/capsule contains:

| | |
|---|---|
| Hydroxychloroquine | 100 mg |
| Linagliptin | 5 mg |
| Excipients q.s. | to make 200 mg tablet/capsule |

ii Each tablet/capsule contains:

| | |
|---|---|
| Hydroxychloroquine | 200 mg |
| Linagliptin | 5 mg |
| Excipients q.s. | to make 300 mg tablet/capsule |

iii. Each tablet/capsule contains:

| | |
|---|---|
| Hydroxychloroquine | 300 mg |
| Linagliptin | 5 mg |
| Excipients q.s. | to make 400 mg tablet/capsule |

iv. Each tablet/capsule contains:

| | |
|---|---|
| Hydroxychloroquine | 400 mg |
| Linagliptin | 5 mg |
| Excipients q.s. | to make 500 mg tablet/capsule |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A pharmaceutical combination comprising (a) hydroxychloroquine or a pharmaceutically acceptable salt thereof; and (b) a DPP-IV inhibitor or a pharmaceutically acceptable salt, solvate, or a prodrug thereof.

2. The pharmaceutical combination according to claim 1, wherein combination is suitable for simultaneous, separate or sequential, including in alternation, or combined use.

3. The pharmaceutical combination according to claim 1, wherein components (a) and (b) are present in one single dosage form or each in separate dosage form.

4. The pharmaceutical combination according to claim 1, in the form of a fixed dose combination.

5. The pharmaceutical combination according to claim 4, wherein the hydroxychloroquine is in a range from 100 mg to 500 mg calculated based on the weight of hydroxychloroquine free base and the DPP-IV inhibitor is in the range from 2 mg to 100 mg calculated based on the weight of the DPP-IV inhibitor free base.

6. The pharmaceutical combination according to claim 1, wherein the DPP-IV inhibitor is selected from the group consisting of sitagliptin, vildagliptin, saxagliptin, melogliptin, P93/01 (Prosidion), alogliptin, denagliptin, Roche 0730699, TS021 (Taisho), E3024 (Eisai), linagliptin, carmegliptin, gosogliptin, teneligliptin, dutogliptin, and a pharmaceutically acceptable salt, solvate, and prodrug thereof.

7. The pharmaceutical combination according to claim 1, wherein the combination is formulated into a dosage form.

8. The pharmaceutical combination according to claim 7, wherein the dosage form is selected from the group consisting of oral, rectal, transdermal, transmucosal, intestinal, and parenteral administration.

9. The pharmaceutical combination according to claim 7, wherein the dosage form is an immediate release formulation, a modified or controlled release formulation, or an extended or sustained release formulation.

10. The pharmaceutical combination according to claim 1, for preventing, slowing the progression of, delaying, improving, restoring, or treating the diseases or conditions resulting from metabolic disorders.

11. The pharmaceutical combination according to claim 1, comprising (a) hydroxychloroquine; and (b) sitagliptin, or a pharmaceutically acceptable salt, solvate, or a prodrug thereof.

12. The pharmaceutical combination according to claim 11, wherein the hydroxychloroquine is in a range from 100 mg to 500 mg calculated based on the weight of hydroxychloroquine free base.

13. The pharmaceutical combination according to claim 11, wherein the sitagliptin is in a range from 3.5 mg to 80 mg calculated based on the weight of sitagliptin free base.

14. The pharmaceutical combination according to claim 11, wherein the sitagliptin is in a range from 6 mg to 31 mg calculated based on the weight of sitagliptin free base.

15. The pharmaceutical combination according to claim 1, further comprising one or more pharmaceutically acceptable carriers or diluents.

16. A method of preventing, slowing the progression of, delaying, improving, restoring, or treating the diseases resulting from metabolic disorders comprising the step of administrating to a subject in need thereof (a) hydroxychloroquine or a pharmaceutically acceptable salt thereof; and (b) a DPP-IV inhibitor or a pharmaceutically acceptable salt, solvate, or a prodrug thereof.

17. The method according to claim 16, wherein the administrating step comprises simultaneous, separate, or sequentia administration of effective amounts of components (a) and (b), in a ratio which provides an additive and/or synergistic effect.

18. The method according to claim 16, wherein the diseases resulting from metabolic disorders are type-1 diabetes mellitus, type-2 diabetes mellitus, central obesity, dyslipidemia, elevated blood pressure, insulin resistance or impaired glucose tolerance (IGT), metabolic syndrome, conditions of impaired fasting and/or post-prandial plasma glucose, metabolic acidosis, ketosis, arthritis, osteoporosis, obesity, prothrombotic state, pro-inflammatory conditions, or combinations thereof.

19. The method according to claim 16, for preventing, slowing the progression of, delaying, improving, restoring or treating a condition or disorder selected from the group consisting of complications of diabetes, cataracts, micro- and macrovascular diseases, and combinations thereof.

20. The method according to claim 16, for preventing, slowing, delaying the progression of, improving, restoring or treating the degeneration of pancreatic beta cells and/or the decline of the functionality of pancreatic beta cells.
